SEQUENCE LISTING (4 sequences)

SEQ ID NO: 1 - DNA, 717 bp, Achromobacter denitrificans NBRC 15125
atgctgagcaagagcctgaccttgaccgaacaggtcgcccgccagatcgcgggcgacatcgccgaaggcgtccattccgtgggcgccaagctgccgcccggccgtgtcctggcggagcagtacggtgtgagcgccgcggtcatccgcgaggccaccgagcgcctgcgcgcccaggggctgatccagagccgccagggctcgggcagcgtggtggtgtcccgcaccggtgctcagggcttccaggtttccgccggcctcgacgatcgcgagcagctggccagcgtctacgaattgcggatggaactggaaggcggcgcggccgccctggcggcgaggcgccgcaacgccaccgaccttgcggccatggccgaggccctggccgcgctggaagcgaacctggaccatccggaacagggcgtcgagcacgacatcgccttccacgtcgccatcgccgccgccacgcacaaccgttattaccaggacctgctgcagtacctgaacctgcagctgcgcctggccgtcagcaccgcgcgcaccaacagccgccgtcaggagggcctgaccgcggtggtgcaccaggaacacgtggccgtctacgacgccatcctcgcgggcgatcccgaccgcgcccgactggcggcgacccgccacttgcagcaggcggccagccgcctgcgtctcgatctcctctctccggccgcaaggcagacatcatga

SEQ ID NO: 2 - DNA, 81 bp, Achromobacter denitrificans NBRC 15125
gagtcgcggcggcgcgccggatccgggctgtcattgtcaaaagttatcagataacctgaaaagtaggcctataatcggcgc

SEQ ID NO: 3 - DNA, 81 bp, Artificial sequence; identical to SEQ ID NO: 2
  The nucleotide sequence of the biotin-tagged dhdO fragment

SEQ ID NO: 4 - DNA, 81 bp, Artificial sequence
  The nucleotide sequence of the biotin-tagged dhdO-1 fragment
gagtcgcggcggcgcgccggatccgggctgtcattgtcaaaatttatcagataacctgaaaagtaggcctataatcggcgc

(19) United States
(12) Patent Application Publication
(10) Pub. No.: US 2024/0077423 A1
(43) Pub. Date: Mar. 7, 2024

(54) A TRANSCRIPTIONAL REGULATOR SPECIFICALLY RESPONDING TO D-2-HYDROXYGLUTARATE AND APPLICATION THEREOF

(71) Applicant: SHANDONG UNIVERSITY, Qingdao, Shandong (CN)

(72) Inventors: Chao GAO, Qingdao (CN); Dan XIAO, Qingdao (CN); Wen ZHANG, Qingdao (CN); Cuiqing MA, Qingdao (CN); Ping XU, Qingdao (CN)

(73) Assignee: SHANDONG UNIVERSITY, Qingdao, Shandong (CN)

(21) Appl. No.: 17/795,351

(22) PCT Filed: Mar. 21, 2021
(86) PCT No.: PCT/CN2021/080525

§ 371 Date: Jul. 26, 2022

(30) Foreign Application Priority Data
Feb. 5, 2021 (CN) 2021101625556

(51) Int. Cl. G01N 21/64; C07K 14/195; C12N 15/70; G01N 21/76

(52) U.S. Cl. CPC G01N 21/6486; C07K 14/195; C12N 15/70; G01N 21/763; C07K 2319/21

(57) ABSTRACT

A transcriptional regulator specifically responding to D-2-hydroxyglutarate (D-2-HG) and its application in the biological detection of D-2-HG. Wherein the transcriptional regulator is named DhdR and the nucleotide sequence is shown as SEQ ID NO: 1. The D-2-HG biosensors B_{D2HG}-0 and B_{D2HG}-1 are constructed using the transcriptional regulator DhdR and can detect biological samples containing D-2-HG.

### CROSS-REFERENCES TO RELATED APPLICATIONS

This application claims priority benefits to Chinese Patent Application No. 202110162555.6, filed 5 Feb. 2021, the contents of which are incorporated herein by reference.

### TECHNICAL FIELD

The present invention relates to a transcriptional regulator and its application, in particular to a transcriptional regulator specifically responding to D-2-hydroxyglutarate and its application in biological detection of D-2-hydroxyglutarate, belonging to the field of biological detection.

### BACKGROUND

D-2-Hydroxyglutarate (D-2-HG) has traditionally been considered an abnormal metabolite associated with the neurometabolic disorder D-2-hydroxyglutaric aciduria (D-2-HGA)^{[1]}. The accumulation of D-2-HG is also caused by mutations in isocitrate dehydrogenase in a variety of tumor cells such as glioma, acute leukemia, chondrosarcoma, and cholangiocarcinoma^{[2]}. In addition, D-2-HG is also an important intermediate metabolite in L-serine synthesis, lysine catabolism, and 4-hydroxybutyric acid catabolism^{[3-5]}, which can be catabolized to 2-ketoglutarate (2-KG) by D-2-hydroxyglutarate dehydrogenase (D2HGDH). However, the regulatory mechanism of D-2-HG metabolism has not been elucidated. As a structural analogue of 2-KG, D-2-HG can also competitively inhibit the activity of 2-ketoglutarate-dependent dioxygenase^{[6]}. D-2-HG is at low concentrations under normal physiological conditions. However, the accumulation of D-2-HG can affect normal life activities. Therefore, the development of D-2-HG detection methods is of great importance for the diagnosis and detection of D-2-HGA and several cancers.

At present, the common methods of detecting D-2-HG mainly include GC-MS/MS and LC-MS/MS^{[7-8]}, which are time-consuming, labor-intensive, and usually require the use of suitable derivatization reagent to make D-2-HG is distinguished from its chiral isomer L-2-hydroxyglutarate (L-2-HG)^{[8]}, limiting the development of diagnostic and therapeutic techniques related to D-2-HG. Bacteria have evolved transcriptional regulators capable of sensing a variety of small molecules, which typically consist of a DNA binding domain and a ligand binding domain. The specific binding of small molecules to ligand binding domains can induce conformational changes of the transcriptional regulator, thereby enhancing or attenuating the interaction between the DNA binding domain of the transcriptional regulator and the DNA binding site on which it acts^{[9]}. Several bacterial transcriptional regulators have been identified and widely used in the quantification of related compounds in various types of samples^{[10-12]}

Alpha technology is a high-sensitivity homogeneous detection technology based on microbeads. At present, it has been reported that transcriptional regulators are used as biorecognition elements in combination with Alpha technology to develop biosensors for high-sensitivity detection of uric acid and oxytetracycline^{[13]}. In view of this, the basis of developing biosensors based on Alpha technology is to screen transcriptional regulators that specifically respond to D-2-HG, and use them as biorecognition elements to develop highly sensitive D-2-HG biosensors. However, the transcriptional regulators that specifically respond to D-2-HG and the biological detection methods of D-2-HG based on transcriptional regulators have not been reported.

### REFERENCES

[1] Kranendijk, M., Struys, E. A., Salomons, G. S., Van der Knaap, M. S. & Jakobs, C. Progress in understanding 2-hydroxyglutaric acidurias. J. Inherit. Metab. Dis. 35, 571-587 (2012).
[2] Ye, D., Guan, K. L. & Xiong, Y. Metabolism, Activity, and Targeting of D- and L-2-Hydroxyglutarates. Trends Cancer 4, 151-165 (2018).
[3] Zhang, W. et al. Coupling between D-3-phosphoglycerate dehydrogenase and D-2-hydroxyglutarate dehydrogenase drives bacterial L-serine synthesis. Proc. Natl. Acad. Sci. USA 114, E7574-E7582 (2017).
[4] Araújo, W. L. et al. Identification of the 2-hydroxyglutarate and isovalery L-CoA dehydrogenases as alternative electron donors linking lysine catabolism to the electron transport chain of Arabidopsis mitochondria. Plant Cell 22, 1549-1563 (2010).
[5] Kaufman, E. E., Nelson, T., Fales, H. M. & Levin, D. M. Isolation and characterization of a hydroxyacid-oxoacid transhydrogenase from rat kidney mitochondria. J. Biol. Chem. 263, 16872-16879 (1988).
[6] Xu, W. et al. Oncometabolite 2-hydroxyglutarate is a competitive inhibitor of α-ketoglutarate-dependent dioxygenases. Cancer Cell 19, 17-30 (2011).
[7] Fernández-Galán, E. et al. Validation of a routine gas chromatography mass spectrometry method for 2-hydroxyglutarate quantification in human serum as a screening tool for detection of idh mutations. J. Chromatogr. B. Analyt. Technol. Biomed. Life Sci. 1083, 28-34 (2018).
[8] Struys, E. A., Jansen, E. E., Verhoeven, N. M. & Jakobs, C. Measurement of urinary D- and L-2-hydroxyglutarate enantiomers by stable-isotope-dilution liquid chromatography-tandem mass spectrometry after derivatization with diacetyL-L-tartaric anhydride. Clin. Chem. 50, 1391-1395 (2004).
[9] Libis, V., Delépine, B. & Faulon, J. L. Sensing new chemicals with bacterial transcription factors. Curr. Opin. Microbiol. 33, 105-112 (2016).
[10] Cao, J. et al. Harnessing a previously unidentified capability of bacterial allosteric transcription factors for sensing diverse small molecules in vitro. Sci. Adv. 4, eaau4602 (2018).
[11] Liang, M. et al. A CRISPR-Cas12a-derived biosensing platform for the highly sensitive detection of diverse small molecules. Nat. Commun. 10, 3672 (2019).
[12] Grazon, C. et al. A progesterone biosensor derived from microbial screening. Nat. Commun. 11, 1276 (2020).
[13] Li, S. et al. A platform for the development of novel biosensors by configuring allosteric transcription factor recognition with amplified luminescent proximity homogeneous assays. Chem. Commun. 53, 99-102 (2017).

### SUMMARY

In view of the shortcomings of the existing methods, such as the time-consuming, labor-consuming, and complicated detection process, the present invention is to provide a transcriptional regulator specifically responding to D-2-hydroxyglutarate (D-2-HG) and its application in the biological detection of D-2-HG.

A transcriptional regulator specifically responding to D-2-HG is provided, wherein the transcriptional regulator is named DhdR, with nucleotide sequence shown in SEQ ID NO: 1. DhdR belongs to the transcriptional repressor protein of the GntR family. It is capable of binding with its upstream promoter region and specifically responding to D-2-HG. The binding site of the transcriptional regulator DhdR acted on its promoter region is 5′-AAAGTTATCAGATAACCTGAAAAGTAG-3′. When D-2-HG is present, it will combine with the transcriptional regulator DhdR and induce the conformational changes of DhdR, resulting in the dissociation of the transcriptional regulator DhdR and the target DNA acted by the transcriptional regulator DhdR.

The proof that the transcriptional regulator DhdR can respond specifically to D-2-HG is shown as:
(1) The pETDuet-1 vector is used to exogenously express the transcriptional regulator dhdR, wherein the nucleotide sequence of the transcriptional regulator DhdR is shown in SEQ ID NO: 1.

Then, the recombinant plasmid pETDuet-dhdR is constructed and introduced into the expression strain Escherichia coli BL21(DE3). The recombinant strain is cultured to OD_{600 nm} of 0.6-0.8, induced by adding IPTG, and purified by using nickel column affinity chromatography to obtain the His-tagged DhdR protein;
(2) The target DNA containing the promoter region acted by the transcriptional regulator DhdR is amplified and purified by PCR and named dhdO; wherein, the nucleotide sequence of the target DNA fragment dhdO is shown in SEQ ID NO: 2;
(3) The function of the transcriptional regulator DhdR is confirmed by electrophoretic mobility shift assays.

DhdR is incubated with different compounds (D-2-HG, L-2-HG, D-malate, D-lactate, glutarate, 2-ketoglutarate, or pyruvate) respectively. Then, the target DNA fragment dhdO is added. The influence on the binding capacity of DhdR and the target DNA fragment dhdO in the presence of the different compounds is analyzed through electrophoresis separation, staining, and imaging. The experiments show that only D-2-HG prevents the binding of DhdR and the target DNA fragment dhdO, DhdR is demonstrating that DhdR is a transcriptional regulator that specifically responds to D-2-HG.

The present invention provides the transcriptional regulator that specifically responds to D-2-HG and its application in the biological detection of D-2-HG.

A biosensor for the detection of D-2-HG is constructed by using a transcriptional regulator DhdR specifically responding to D-2-HG, wherein the biosensor is composed of His-tagged DhdR protein, biotinylated dhdO or biotinylated dhdO-1, streptavidin-coated donor beads and nickel-chelated acceptor beads; the biosensor is capable of producing obvious luminescence signals at 520-620 nm upon laser excitation at 680 nm. When the biosensor detects the presence of D-2-HG in the sample, the conformational change of DhdR is induced due to the binding of D-2-HG to DhdR protein, which leads to the dissociation of the originally bound transcriptional regulator DhdR from the biotinylated dhdO. Finally, the distance between the donor beads and the acceptor beads is increased, resulting in decreased luminescence signals.

Wherein, the obtaining of the His-tagged DhdR protein comprising: the pETDuet-1 vector is used to exogenously express the GntR family transcriptional regulator dhdR from Achromobacter denitrificans NBRC 15125. The nucleotide sequence of DhdR is shown in SEQ ID NO: 1. and then a recombinant plasmid pETDuet-dhdR is constructed and introduced into the expression strain E. coli BL21(DE3) by heat-stimulated transformation. Then the recombinant expression strain is cultured at 37° C. and 180 rpm to an OD_{600 nm} of 0.6-0.8, induced with 1 mM IPTG at 16° C. and 160 rpm for 12 hours, and purified by nickel column affinity chromatography to obtain the His-tagged DhdR protein.

The nucleotide sequence of the biotinylated dhdO fragment is shown in SEQ ID NO: 3. The unlabeled dhdO fragment is obtained by recombinant PCR using Bio-dhdO upstream primers and Bio-dhdO downstream primers; the unlabeled dhdO fragment is then used as a template, and the biotinylated dhdO fragment is obtained by PCR amplification using Bio upstream primers and Bio-dhdO downstream primers, and purified by gel extraction kit, wherein the sequence of PCR primers are as follows:

Bio-dhdO upstream primer:
5′-GAGTCGCGGCGGCGCGCCGGATCCGGGCTGTCATTGTCA-3′;
Bio-dhdO downstream primer:
5′-GCGCCGATTATAGGCCTACTTTTCAGGTTATCTGATAACTTTTGAC
AATGACAGCCCGGAT-3′;
Bio upstream primer:
5′-GAGTCGCGGCGGCGCGCCGGAT-3′,
biotin is modified at the 5′ end.

The nucleotide sequence of the biotinylated dhdO-1 fragment is shown in SEQ ID NO: 4, and the fragment is prepared by mutating the 43rd base “G” of the biotinylated dhdO fragment into a base “T”; the unlabeled dhdO-1 fragment is obtained by recombinant PCR using Bio-dhdO upstream primers and Bio-dhdO-1 downstream primers; the unlabeled dhdO-1 fragment is then used as a template, the biotinylated dhdO-1 fragment is obtained by PCR amplification using Bio upstream primers and Bio-dhdO-1 downstream primers, and purified and recovered by gel extraction kit; wherein the Bio-dhdO upstream primers and Bio upstream primers for amplifying the biotinylated dhdO-1 fragment are the same as the primer for amplifying the biotinylated dhdO fragment, and the sequence of the Bio-dhdO-1 downstream primer is as follows:

Bio-dhdO-1 downstream primer:
5′-GCGCCGATTATAGGCCTACTTTTCAGGTTATCTGATAAATTTTGAC
AATGACAGCCCGGAT-3′.

The streptavidin-coated donor beads and the nickel-chelated acceptor beads are purchased from PerkinElmer (USA).

Preferred embodiments of the aforementioned biosensor for detecting D-2-HG are provided as:

The biosensor consists of 1 nM biotinylated dhdO fragment, 0.3 nM DhdR protein, 20 μg/mL streptavidin-coated donor beads, and 20 μg/mL nickel-chelated acceptor beads, and is named D-2-HG biosensor B_{D2HG}-0; alternatively, the biosensor consists of 1 nM biotinylated dhdO-1 fragment, 0.3 nM DhdR protein, 20 μg/mL streptavidin-coated donor beads, and 20 μg/mL nickel-chelated acceptor beads, and is named D-2-HG biosensor B_{D2HG}-1.

A method for preparing the biosensor for detecting D-2-HG comprises the following steps:
(1) preparing a His-tagged DhdR protein;
(2) preparing a biotinylated dhdO fragment or a biotinylated dhdO-1 fragment;
(3) combining the His-tagged DhdR protein, the biotinylated dhdO fragment, or the biotinylated dhdO-1 fragment with commercially available streptavidin-coated donor beads and nickel-chelated acceptor beads to obtain the D-2-HG biosensor.

Provided is an application of the biosensor for detection of D-2-HG based on specific transcriptional regulator DhdR described by the present invention in detecting biological samples containing D-2-HG.

Wherein the D-2-HG biosensor is preferably B_{D2HG}-1.

The method for detecting biological samples containing D-2-HG is as follows:

D-2-HG solutions in gradient concentrations were prepared using healthy adult serum, urine, and cell culture medium as different types of biological samples, respectively. The dose-response curves and quantitative results for D-2-HG in the different types of biological samples were determined using the D-2-HG biosensor B_{D2HG}-1.

Wherein the method of determining the dose-response curves of different types of biological samples comprises: 1 nM biotinylated dhdO-1 fragment, 0.3 nM DhdR protein, and equal volumes of a solution containing different concentrations of D-2-HG are added into a white 384-well plate, mixed evenly and incubated for 30 minutes; 20 μg/mL acceptor beads are added and incubated for 30 minutes; 20 μg/mL donor beads are added and incubated for 60 minutes. The incubation is carried out at room temperature and protected from light (in dark). The luminescence signal for each sample is measured by EnSight Multimode Plate Reader. The excitation wavelength is 680 nm and the detection wavelength is 520-620 nm. The background signal without a D-2-HG biosensor is deducted at each emission wavelength, and the dose-response curves for D-2-HG in different types of biological samples are obtained.

Wherein the method for determining quantitative results of D-2-HG concentrations in different types of biological samples comprises: determining the luminescence signals of different types of samples at 50 μM, 150 μM, 500 μM, 1500 μM, and 3500 μM using the above method of determining the dose-response curves, subtracting the background signal without D-2-HG biosensor; using the above dose-response curves for D-2-HG in different types of biological samples to calculate the luminescence signal values of the samples after subtracting the background signals to the concentrations of D-2-HG and multiplying them by the corresponding dilutions to obtain the quantitative results for D-2-HG in different types of biological samples.

In the embodiment of the present invention, all the prepared samples can be diluted to the desired concentration using an HBS-P buffer, and the ingredients of the HBS-P buffer are as follows: 10 mM HEPES, 150 mM NaCl, 0.1% BSA, 0.005% Tween-20, pH 7.4.

The D-2-HG biosensor based on the specific transcriptional regulator DhdR provided by the present invention combines the transcriptional regulator DhdR and its target DNA dhdO with the commercial Alpha kit based on microbeads developed by PerkinElmer Company. It can convert and amplify the biological concentration of D-2-HG into luminescence signals. Upon laser excitation at 680 nm, the photosensitizer in donor beads converts the ambient oxygen to singlet oxygen. During the half-life of 4 μs, the singlet oxygen may diffuse approximately 200 nm in solution, allowing energy transfer from the singlet oxygen to the thioxene derivatives within the acceptor beads, which eventually generates extensive luminescence signals at 520-620 nm. When the biosensor detects the presence of D-2-HG, D-2-HG induces the conformational changes of DhdR by binding to the transcriptional regulator DhdR, which can lead to dissociation of the originally bound transcriptional regulator DhdR from the biotinylated dhdO. Therefore, the distance between the donor beads and the acceptor beads increases, resulting in reduced luminescence signals. The intensity of the luminescence signals is related to the concentration of D-2-HG detected. When the concentrations of D-2-HG are high, the luminescence signals are weakened.

The outstanding features and beneficial effects of the present invention are shown as:

(1) The transcriptional regulator DhdR provided by the present invention, which specifically responds to D-2-HG, is derived from Achromobacter denitrificans NBRC 15125. It is the first transcriptional regulator identified to regulate the catabolism of D-2-HG and specifically respond to D-2-HG. DhdR can bind to its target DNA dhdO under normal conditions. When D-2-HG is present, it will combine with the transcriptional regulator DhdR and induce the conformational changes of the DhdR, resulting in the dissociation of the original combined transcriptional regulator DhdR and the target DNA dhdO. The intensity of luminescence signals of the D-2-HG biosensor based on specific transcriptional regulator DhdR is related to the concentration of D-2-HG detected. The concentration of D-2-HG can be detected by using the characteristic that the increase in the distance between the donor beads and the acceptor beads can lead to the luminescence signals reduce.

(2) The D-2-HG biosensor based on the specific transcriptional regulator DhdR provided by the present invention combines DhdR (a transcriptional regulator specifically responding to D-2-HG) and its target DNA dhdO with a commercial Alpha kit, and converts D-2-HG concentration signal into luminescence signals for output, which is simple in composition, easy to prepare, highly specific in detection, and convenient in operation.

(3) The D-2-HG biosensor based on the specific transcriptional regulator DhdR provided by the present invention is suitable for quantifying D-2-HG in different types of biological samples such as serum, urine, and cell culture medium, and the quantitative results are consistent with those of the traditional detection method LC-MS/MS. It realizes the high sensitivity and specificity of D-2-HG detection in different types of biological samples and has broad application prospects in the diagnosis and treatment of D-2-HG-related diseases and the development of targeted drugs.

### BRIEF DESCRIPTION OF THE DRAWINGS

The accompanying drawings, which are incorporated in and constitute a part of this application, are included to provide a further understanding of the application, and the description of the exemplary embodiments and illustrations of the application are intended to explain the application and are not intended to limit the application.

FIG. 1: SDS-PAGE analysis of purified DhdR.

FIG. 2: Specificity analysis of DhdR for D-2-HG.

FIG. 3: Schematic diagram of the principle of detecting D-2-HG based on DhdR.

FIG. 4: Dose-response curve of B_{D2HG}-0 for D-2-HG.

FIG. 5: Dose-response curve of B_{D2HG}-1 to D-2-HG.

FIG. 6: Comparison between the quantitative results of D-2-HG in serum by B_{D2HG}-1 and LC-MS/MS.

FIG. 7: Comparison between the quantitative results of D-2-HG in urine by B_{D2HG}-1 and LC-MS/MS.

FIG. 8: Comparison between the quantitative results of D-2-HG in cell culture medium by B_{D2HG}-1 and LC-MS/MS.

FIG. 9: Detection of D-2-HG levels in HT1080 cell culture medium supernatant by B_{D2HG}-1.

### DETAILED DESCRIPTION

The following is a detailed description of the contents of the present application in conjunction with the specific accompanying drawings and examples. It should be noted that the following description is intended only to explain the invention, not to limit it in any way, and that any simple modifications, equivalent changes, and modifications to the embodiment based on the technical substance of the invention are within the scope of the technical solution of the invention.

In the following examples, the denitrifying Achromobacter denitrificans NBRC 15125 is purchased from BeNa Culture Collection (BNCC) with a strain serial number NCTC 8582. The expression vector pETDuet-1 was purchased from Novagen. The Alpha kit was purchased from PerkinElmer with item number 6760619, which contained streptavidin-coated donor beads and nickel-chelated acceptor beads. Other materials and reagents used, unless otherwise specified, were obtained from commercial sources. The experimental methods used, which are not specifically described, are conventional methods.

### Example 1: Acquisition and Identification of a Transcriptional Regulator DhdR Specifically Responding to D-2-HG

The media and reagents used in this example are as follows:

Luria-Bertani (LB) medium: 0.5% yeast powder, 1% peptone, 1% NaCl

Loading buffer: 20 mM Na₂HPO₄, 20 mM imidazole, 500 mM NaCl, pH 7.4.

Elution Buffer: 20 mM Na₂HPO₄, 500 mM imidazole, 500 mM NaCl, pH 7.4.

Binding buffer: 10 mM Tris-HCl, 50 mM KCl, 0.5 mM EDTA, 10% glycerol, 1 mM dithiothreitol, pH 7.4.

Electrophoretic mobility shift assays buffer: 89 mM Tris, 89 mM boric acid, 2 mM EDTA, pH 8.3.

(1) Expression and Purification of DhdR

DhdR used in the present invention is a repressor protein derived from Achromobacter denitrificans NBRC 15125. The dhdR nucleotide fragment was obtained by PCR amplification using the genome of Achromobacter denitrificans NBRC 15125, and the dhdR nucleotide fragment and pETDuet-1 plasmid were digested using SacI/HindIII restriction endonuclease, and the recombinant plasmid pETDuet-dhdR was obtained by ligation with T4 DNA ligase. The recombinant plasmid was transformed into the competent cell E. coli BL21(DE3) by heat-stimulated transformation. The resulting cells were incubated for 50 min at 37° C. in a shaker, plated on LB solid medium (containing 100 μg/mL ampicillin), and incubated for 12 h at 37° C. in an incubator. The single colonies were picked for PCR validation.

Wherein, the primer sequences for amplifying dhdR nucleotide fragments were as follows:

dhdR upstream primer:
5′-ATATGAGCTCGATGAGCGCATCCGACTTTA-3′,
carrying a SacI restriction site;
dhdR downstream primer:
5′-TATTAAGCTTCTACAGCAGCTTCCCGGGAT-3′,
carrying a HindIII restriction site.

The DNA polymerase used in the PCR is TransStart FastPfu DNA polymerase purchased from TransGen Biotech (Beijing), and the PCR reaction is performed according to the instructions of the manufacturer of DNA polymerase.

Correctly validated strains were inoculated in 1 L of LB liquid medium (containing 100 μg/mL ampicillin) and incubated at 37° C. with 180 rpm to an OD_{600 nm} of approximately 0.6. Then, the cell culture was induced with 1 mM IPTG at 16° C. and 160 rpm for 12 hours. The cells were collected by centrifugation at 4° C. and 6,000 rpm for 10 minutes, resuspended to an OD_{600 nm} of approximately 25 in loading buffer containing 1 mM PMSF and 10% glycerol and lysed by using a high-pressure cell disrupter. The supernatant was collected by centrifugation at 4° C. and 12,000 rpm for 50 minutes.

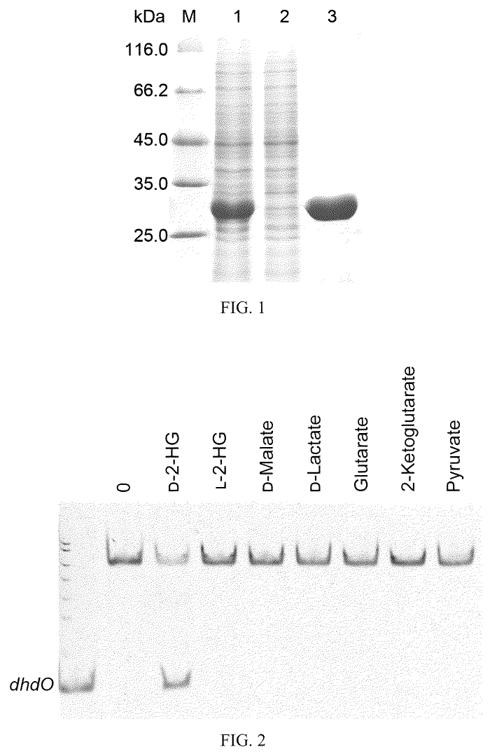

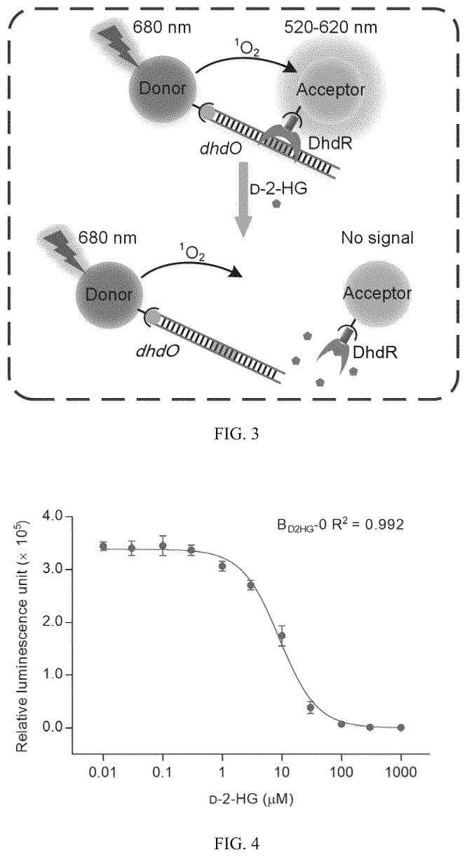

The supernatant was then loaded into a 5 mL HisTrap HP nickel affinity chromatography column and gradient elution was performed using an elution buffer. The purity of His₆-DhdR was analyzed by sodium dodecyl sulfate-polyacrylamide gel electrophoresis (SDS-PAGE), and the results were shown in FIG. 1. The purified His₆-DhdR was concentrated in an ultrafiltration tube, added with 10% glycerol and stored at +80° C.

(2) Acquisition of Target DNA Fragment dhdO

The target DNA fragment dhdO was obtained by PCR amplification using the genome of Achromobacter denitrificans NBRC 15125 as a template. The amplified dhdO nucleotide fragment was purified and recovered using a gel extraction kit, and the DNA concentration was determined by NanoDrop ND-1000; wherein the primer sequences for amplifying the dhdO nucleotide fragment were as follows:

dhdO upstream primer:
5′-GAGTCGCGGCGGCGCGCCGGAT-3′;
dhdO downstream primer:
5′GCGCCGATTATAGGCCTACTT3′.

The DNA polymerase used in the PCR reaction is TransStart FastPfu DNA polymerase purchased from TransGen Biotech (Beijing), and the PCR reaction is performed according to the manufacturer of the DNA polymerase.

(3) Analysis of DhdR Effector by Electrophoretic Mobility Shift Assays

The dhdO nucleotide fragment obtained in step (2) above was diluted to 100 nM using binding buffer, and the purified DhdR protein was diluted to 2000 nM. 70 nM purified DhdR protein was first incubated with 40 mM different compounds (D-2-HG, L-2-HG, D-malate, D-lactate, glutarate, 2-ketoglutarate, or pyruvate) at 30° C. for 15 minutes in 18 μL binding buffer. Subsequently, 2 μL of 100 nM dhdO was added and incubated at 30° C. for another 30 min. 10 μL of the sample was analyzed by electrophoresis with 6% native polyacrylamide at 170 V for 45 min on ice. The gel was then stained with SYBR Green I for 30 min avoiding light and photographed by gel imager. As shown in FIG. 2, only D-2-HG could inhibit the binding of DhdR to dhdO, and other compounds could not interfere with the binding of DhdR to dhdO, indicating that DhdR specifically responded to D-2-HG. DhdR can bind to dhdO in the absence of D-2-HG. However, when D-2-HG is present, DhdR will be dissociated from dhdO.

### Example 2: Construction of Biosensor B_{D2HG}-0 Using Transcriptional Regulator DhdR Specifically Responsive to D-2-HG

The reagents used in this example are as follows:

HBS-P buffer: 10 mM HEPES, 150 mM NaCl, 0.1% BSA, 0.005% Tween-20, pH 7.4.

(1) Amplification and Purification of Biotinylated dhdO Fragment

Biotinylated dhdO fragment was obtained by two rounds of PCR:

In the first round, the unlabeled dhdO fragment was amplified by overlap PCR using Bio-dhdO upstream primers and Bio-dhdO downstream primers. In the second round, the unlabeled dhdO fragment, which was the product of the first round PCR, was used as the template, and the biotinylated dhdO fragment was amplified using primers Bio upstream primers and Bio-dhdO downstream primers. The amplified biotinylated dhdO fragment was purified and recovered by gel extraction kit, and the DNA concentration was determined by NanoDrop ND-1000.

Wherein, the primer sequences for amplifying biotinylated dhdO fragment were as follows:

Bio-dhdO upstream primer:
5′GAGTCGCGGCGGCGCGCCGGATCCGGGCTGTCATTGTCA-3′;
Bio-dhdO downstream primer:
5′-GCGCCGATTATAGGCCTACTTTTCAGGTTATCTGATAACTTTTGAC
AATGACAGCCCGGAT-3′;
Bio upstream primer:
5′-GAGTCGCGGCGGCGCGCCGGAT-3′,
biotin is modified at the 5′ end.

The DNA polymerase used in the PCR reaction is TransStart FastPfu DNA polymerase purchased from TransGen Biotech (Beijing), and the PCR reaction is performed according to the manufacturer of the DNA polymerase.

(2) Detection of the Luminescence Signal

5 μL biotinylated dhdO fragment, 5 μL DhdR protein, and 5 μL solution containing D-2-HG were added into a white 384-well plate, mixed evenly by the oscillation function of a microplate reader, and incubated in dark at room temperature for 30 minutes. Then, 5 μL acceptor beads (20 μg/mL) were added and incubated in dark at room temperature for 30 minutes; Finally, 5 μL donor beads (20 μg/mL) were added and incubated in dark at room temperature for 60 minutes. The luminescence signal was measured by EnSight Multimode Plate Reader. The excitation wavelength was 680 nm and the detection wavelength was 520-620 nm. The background signal without D-2-HG biosensor was deducted at each emission wavelength.

(3) Response of D-2-HG Biosensor B_{D2HG}-0 to D-2-HG

According to the method of detecting the luminescence signal described in (2), a gradient D-2-HG solution dissolved in HBS-P buffer was used and mixed with 5 μL of 1 nM biotinylated dhdO fragment, 0.3 nM DhdR protein, donor beads, and acceptor beads. The luminescence signal was detected to plot the dose-response curves. As shown in FIG. 4, the luminescence signal responded to D-2-HG in a concentration-dependent manner.

The combination of the 1 nM biotinylated dhdO fragment, 0.3 nM DhdR protein, 20 μg/mL donor beads, and 20 μg/mL acceptor beads was named as D-2-HG biosensor B_{D2HG}-0, with a limit of detection of 0.8 μM and a detection range of about 2-50 μM.

### Example 3: Construction of Biosensor B_{D2HG}-1 Using Transcriptional Regulator DhdR Specifically Responsive to D-2-HG

The reagents used in this example are as follows:

HBS-P buffer: 10 mM HEPES, 150 mM NaCl, 0.1% BSA, 0.005% Tween-20, pH 7.4.

(1) Amplification and Purification of Biotinylated dhdO-1 Fragment

The biotinylated dhdO-1 fragment was obtained by two rounds of PCR:

In the first round, the unlabeled dhdO-1 fragment was amplified by overlap PCR using Bio-dhdO upstream primers and Bio-dhdO-1 downstream primers. In the second round, the unlabeled dhdO-1 fragment, which was the product of the first-round PCR, was used as the template, and the biotinylated dhdO-1 fragment was amplified using primers Bio upstream primers and Bio-dhdO-1 downstream primers. The amplified biotinylated dhdO-1 fragment was purified and recovered by gel extraction kit, and the DNA concentration was determined by NanoDrop ND-1000, wherein the primer sequences for amplifying the biotinylated dhdO-1 fragment were as follows:

Bio-dhdO upstream primer:
5′-GAGTCGCGGCGGCGCGCCGGATCCGGGCTGTCATTGTCA-3′;
Bio-dhdO-1 downstream primer:
5′-GCGCCGATTATAGGCCTACTTTTCAGGTTATCTGATAAATTTTGAC
AATGACAGCCCGGAT-3′;
Bio upstream primer:
5′-GAGTCGCGGCGGCGCGCCGGAT-3′,
biotin is modified at the 5′ end.

The DNA polymerase used in the PCR reaction is TransStart FastPfu DNA polymerase purchased from TransGen Biotech (Beijing), and the PCR reaction is performed according to the manufacturer of the DNA polymerase.

(2) Detection of the Luminescence Signal

5 μL biotinylated dhdO-1 fragment, 5 μL DhdR protein and 5 μL solution containing D-2-HG were added into a white 384-well plate, mixed evenly by the oscillation function of a microplate reader, and incubated in dark at room temperature for 30 minutes. Then, 5 μL acceptor beads (20 μg/mL) were added and incubated in dark at room temperature for 30 minutes. Finally, 5 μL donor beads (20 μg/mL) were added and incubated in dark at room temperature for 60 minutes. The luminescence signal was measured by EnSight Multimode Plate Reader. The excitation wavelength was 680 nm and the detection wavelength was 520-620 nm. The background signal without a D-2-HG biosensor was deducted at each emission wavelength.

(3) Response of D-2-HG Biosensor B_{D2HG}-1 to D-2-HG

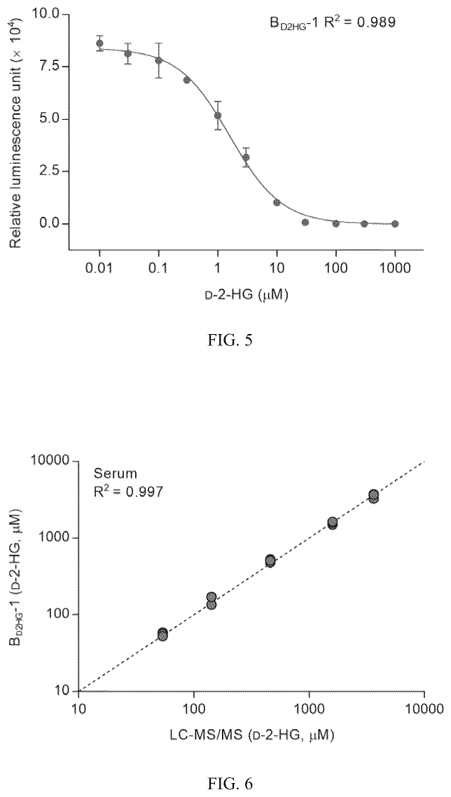

According to the method of detecting the luminescence signal described in (2), a gradient D-2-HG solution dissolved in HBS-P buffer was used and mixed with 5 μL of 1 nM biotinylated dhdO-1 fragment, 0.3 nM DhdR protein, donor beads, and acceptor beads. The luminescence signal was detected to plot the dose-response curves. As shown in FIG. 5, the luminescence signal responded to D-2-HG in a concentration-dependent manner.

The combination of the 1 nM biotinylated dhdO-1 fragment, 0.3 nM DhdR protein, 20 μg/mL donor beads, and 20 μg/mL acceptor beads was named as D-2-HG biosensor B_{D2HG}-1, with a limit of detection of 0.08 μM and a detection range of about 0.3-20 μM. It was superior to D-2-HG biosensor B_{D2HG}-0 and can be preferentially applied to detect the concentration of D-2-HG in vitro.

### Example 4. Application of D-2-HG Biosensor B_{D2HG}-1 in the Detection of Biological Samples Containing D-2-HG

D-2-HG solutions in gradient concentrations were prepared using healthy adult serum, urine, and cell culture medium as different types of biological samples, respectively. The dose-response curves and quantitative results for D-2-HG in different types of biological samples were determined using the D-2-HG biosensor B_{D2HG}-1.

Since the limit of detection of the D-2-HG biosensor B_{D2HG}-1 is 0.08 μM, which is extremely sensitive, only small amounts of samples are required and diluted for the detection of biological samples. The gradient concentration D-2-HG solution was prepared in the following way:
(1) Serum and urine of healthy adults were respectively diluted 100 times with HBS-P buffer solution, and cell culture medium was diluted 10 times with HBS-P buffer solution;
(2) Preparation of 100 mM D-2-HG solution with ddH₂O,
(3) 100 mM D-2-HG solution was gradiently diluted with a 100-fold diluted serum of healthy adults, 100-fold diluted urine, and 10-fold diluted cell culture medium, respectively. The dilution concentration range is 0.05 μM-5000 μM. The final D-2-HG concentration range of plotted dose-response curve is 0.01 μM-1000 μM because the sample was diluted 5 times when added to the system of the biosensor for detection.

The dose-response curves for D-2-HG in different types of biological samples were determined by adding 5 μL of a biotinylated dhdO-1 fragment (1 nM), 5 μL of DhdR protein (0.3 nM), and 5 μL of a solution containing a standard concentration of D-2-HG into a white 384-well plate, mixing them evenly by using the oscillation function of the microplate reader, and incubating them in dark at room temperature for 30 minutes. Then, 5 μL of nickel-chelated acceptor beads (20 μg/mL) was added and incubated in dark for 30 minutes. Finally, 5 μL of streptavidin-coated donor beads (20 μg/mL) was added and incubated in dark for 60 minutes at room temperature. The luminescence signal was measured by EnSight Multimode Plate Reader. The excitation wavelength was 680 nm and the detection wavelength was 520-620 nm. The background signal without a D-2-HG biosensor was deducted at each emission wavelength and the dose-response curves for D-2-HG in different types of biological samples were obtained.

The method for quantification of concentrations of D-2-HG in different types of biological samples was as follows:

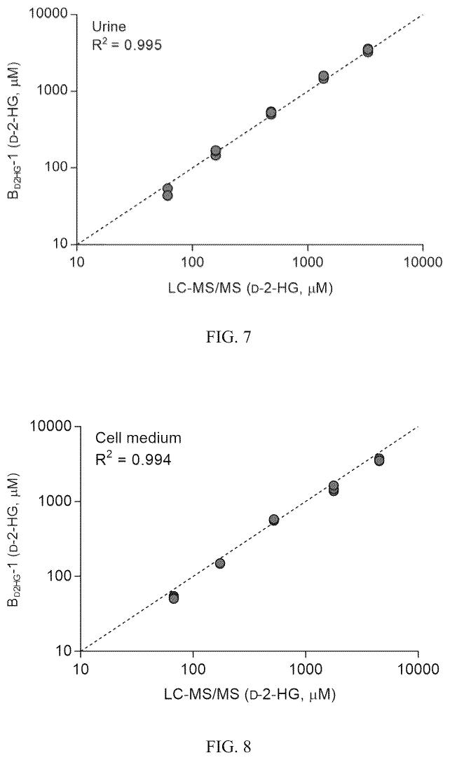

Different types of biological samples with concentrations of 50 μM, 150 μM, 500 μM, 1500 μM, and 3500 μM were prepared by using undiluted healthy adult serum, urine, and cell culture medium respectively. 5 μL biotinylated dhdO-1 fragment (1 nM), 5 μL DhdR protein (0.3 nM), and 5 μL biological sample containing D-2-HG were added into a white 384-well plate, mixed evenly by oscillation function of a microplate reader, and incubated in dark at room temperature for 30 minutes. 5 μL nickel-chelated acceptor beads (20 μg/mL) were added and incubated in dark at room temperature for 30 minutes. 5 μL streptavidin-coated donor beads (20 μg/mL) were added and incubated in dark at room temperature for 60 minutes. The luminescence signal was measured by EnSight Multimode Plate Reader. The excitation wavelength was 680 nm and the detection wavelength was 520-620 nm. The background signal without a D-2-HG biosensor was deducted at each emission wavelength. The quantitative results for D-2-HG in different types of biological samples were obtained by using the above dose-response curves for D-2-HG in different types of biological samples to correspond to the luminescence signal values of the samples after subtracting the background signals to the concentrations of D-2-HG and multiplying them by the corresponding dilutions. The results of the quantification were compared with those of the LC-MS/MS technique. As shown in FIGS. 6-8, the quantification results of the D-2-HG biosensor B_{D2HG}-1 are consistent with those of the LC-MS/MS technique, indicating that D-2-HG biosensor B_{D2HG}-1 is fully applicable to the accurate quantification of D-2-HG in different types of biological samples.

### Example 5: Application of D-2-HG Biosensor B_{D2HG}-1 in the Detection of D-2-HG Concentration in HT1080 Cell Culture Medium Supernatant

The HT1080 cell in the example was purchased from Procell Life Science & Technology Co., Ltd.

HT1080 cells carry the IDH1/R132C mutation; the inhibitor GSK864 inhibits IDH/R132C and AGI-6780 inhibits IDH2/R140Q; 1 mL of each culture medium supernatant of HT1080 cells treated with different inhibitors (GSK864, AGI-6780) was taken as the sample to be detected.

The detection method of D-2-HG in the sample to be detected was as follows:

100 mM D-2-HG solution was diluted with HT1080 cell culture medium diluted 10 times by HBS-P buffer to prepare a standard solution of D-2-HG with a gradient concentration ranging from 0.05 μM to 5000 μM. The dose-response curves with a final D-2-HG concentration range of 0.01 μM-1000 μM were plotted according to the method described in Example 4. 5 μL biotinylated dhdO-1 fragment (1 nM), 5 μL DhdR protein (0.3 nM) and 5 μL biological sample containing D-2-HG were added into a white 384-well plate, mixed evenly by the oscillation function of a microplate reader, and incubated in dark at room temperature for 30 minutes. Then, 5 μL nickel-chelated acceptor beads (20 μg/mL) were added and incubated in dark at room temperature for 30 minutes. Finally, 5 μL streptavidin-coated donor beads (20 μg/mL) were added and incubated in dark at room temperature for 60 minutes. The luminescence signal was measured by EnSight Multimode Plate Reader. The excitation wavelength was 680 nm and the detection wavelength was 520-620 nm. The background signal without a D-2-HG biosensor was deducted at each emission wavelength, and the dose-response curves for the quantification of D-2-HG in HT1080 cells were obtained.

The method for quantification of concentrations of D-2-HG in the sample to be tested was as follows:

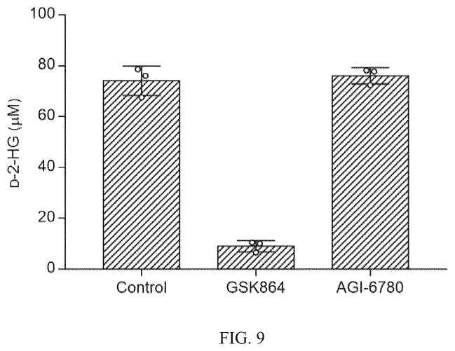

The samples to be tested were diluted in appropriate times using an HBS-P buffer. 5 μL biotinylated dhdO-1 fragment (1 nM), 5 μL DhdR protein (0.3 nM), and 5 μL diluted cell culture medium supernatant were added into a white 384-well plate, mixed evenly by oscillation function of a microplate reader, and incubated in dark at room temperature for 30 minutes. Then, 5 μL nickel-chelated acceptor beads (20 μg/mL) were added and incubated in dark at room temperature for 30 minutes. Finally, 5 μL streptavidin-coated donor beads (20 μg/mL) were added and incubated in dark at room temperature for 60 minutes. The luminescence signal was measured by EnSight Multimode Plate Reader. The excitation wavelength was 680 nm and the detection wavelength was 520-620 nm. The background signal without a D-2-HG biosensor was deducted at each emission wavelength. The quantitative results for D-2-HG in the HT1080 cell culture medium supernatant were obtained by using the above dose-response curves for D-2-HG in the HT1080 cell culture medium to correspond to the luminescence signal values of the samples after subtracting the background signals to the concentrations of D-2-HG and multiplying them by the corresponding dilutions. The results of the quantification were compared with those of the LC-MS/MS technique, and the results were shown in FIG. 9.

1. A transcriptional regulator specifically responding to d-2-hydroxyglutarate (d-2-HG), wherein,
the transcriptional regulator is named DhdR and the nucleotide sequence is shown as SEQ ID NO: 1, DhdR belongs to the transcriptional repressor protein of the GntR family, is capable of combining with the promoter region upstream of the transcriptional regulator and specifically responding to d-2-HG; the binding site of the transcriptional regulator DhdR acted on its promoter region is 5′-AAAGTTATCAGATAACCTGAAAAGTAG-3′; when d-2-HG is present, it will combine with the transcriptional regulator DhdR and induce the conformational change of DhdR, resulting in the dissociation of the transcriptional regulator DhdR and the target DNA acted by the transcriptional regulator DhdR; the target DNA is named dhdO and the nucleotide sequence is shown as SEQ ID NO: 2, dhdO comprises a promoter region acted by the transcriptional regulator DhdR.

2. An application of the transcriptional regulator specifically responding to d-2-HG according to claim 1 in biological detection of d-2-HG.

3. A biosensor for detection of d-2-HG, being constructed by using the transcriptional regulator DhdR specifically responding to d-2-HG according to claim 1, wherein,
the biosensor is comprised of His-tagged DhdR protein, biotinylated dhdO or biotinylated dhdO-1, streptavidin-coated donor beads, and nickel-chelated acceptor beads; the biosensor is capable of producing obvious luminescence signals at 520-620 nm upon laser excitation at 680 nm; when the biosensor detects the presence of d-2-HG in the sample, the conformational change of DhdR is induced due to the binding of d-2-HG to DhdR, which leads to the dissociation of the originally bound transcriptional regulator DhdR from the biotinylated dhdO, finally, the distance between the donor beads and the acceptor beads is increased, resulting in decreased luminescence signals;
wherein, the obtaining of the His-tagged DhdR protein comprising: exogenously expressing the transcriptional regulator dhdR from Achromobacter denitrificans NBRC 15125, which belongs to the GntR family, and the nucleotide sequence is shown as SEQ ID NO: 1, then, constructing a recombinant plasmid pETDuet-dhdR and introducing the recombinant plasmid pETDuet-dhdR into an expression strain E. coli BL21(DE3) by heat-stimulated transformation, culturing the recombinant expression strain at 37° C. and 180 rpm to an OD_{600 nm} of 0.6-0.8, inducing with 1 mM IPTG at 16° C. and 160 rpm for 12 hours, and purifying by nickel column affinity chromatography to obtain the His-tagged DhdR protein;
the nucleotide sequence of biotinylated dhdO fragment is shown as SEQ ID NO: 3; using Bio-dhdO upstream primers and Bio-dhdO downstream primers to obtain unlabeled dhdO fragments by recombinant PCR; then using the unlabeled dhdO fragment as a template, using Bio upstream primers and Bio-dhdO downstream primers to amplify by PCR, and purifying and recovering by gel extraction kit to obtain the biotinylated dhdO fragment; wherein the sequence of the PCR primers comprising:
Bio-dhdO upstream primer: 5′-
GAGTCGCGGCGGCGCGCCGGATCCGGGCTGTCATTGTCA-3′;
Bio-dhdO downstream primer:
5′-GCGCCGATTATAGGCCTACTTTTCAGGTTATCTGATAACTTTTGAC
AATGACAGCCCGGAT-3′;
Bio upstream primer:
5′-GAGTCGCGGCGGCGCGCCGGAT-3′,
being modified with biotin at the 5′ end;
the biotinylated dhdO-1 fragment is shown as SEQ ID NO: 4, which is prepared by mutating the 43rd base “G” of the biotinylated dhdO fragment into a base “T”; using Bio-dhdO upstream primers and Bio-dhdO-1 downstream primers to obtain unlabeled dhdO-1 fragment by recombinant PCR; then using the unlabeled dhdO-1 fragment as a template, using Bio upstream primers and Bio-dhdO-1 downstream primers to amplify by PCR, and purifying and recovering with gel extraction kit to obtain the biotinylated dhdO-1 fragment; wherein the Bio-dhdO upstream primer and the Bio upstream primer for amplifying the biotinylated dhdO-1 fragment are the same as the primer for amplifying the biotinylated dhdO fragment, and the sequence of the Bio-dhdO-1 downstream primer comprising:
Bio-dhdO-1 downstream primer:
5′-GCGCCGATTATAGGCCTACTTTTCAGGTTATCTGATAAATTTTGAC
AATGACAGCCCGGAT-3′.

4. The biosensor for detecting d-2-HG according to claim 3, wherein,
the d-2-HG biosensor being named B_{d2HG}-0, comprising 1 nM biotinylated dhdO fragment, 0.3 nM DhdR protein, 20 μg/mL streptavidin-coated donor beads, and 20 μg/mL nickel-chelated acceptor beads; or,
the d-2-HG biosensor being named B_{d2HG}-1, comprising 1 nM biotinylated dhdO-1 fragment, 0.3 nM DhdR protein, 20 μg/mL streptavidin-coated donor beads, and 20 μg/mL nickel-chelated acceptor beads.

5. A method for preparing the biosensor for detecting d-2-HG according to claim 3, comprising the steps of:
(1) preparing a His-tagged DhdR protein;
(2) preparing a biotinylated dhdO fragment or a biotinylated dhdO-1 fragment;
(3) combining the His-tagged DhdR protein, the biotinylated dhdO fragment, or the biotinylated dhdO-1 fragment with streptavidin-coated donor beads and nickel-chelated acceptor beads to obtain the d-2-HG biosensor.

6. An application of the biosensor for detecting d-2-HG according to claim 3 in detecting biological samples containing d-2-HG.

7. The application according to claim 6, wherein the biosensor for detecting d-2-HG is B_{d2HG}-1.

8. The application according to claim 7, wherein the method for detecting a biological sample containing d-2-HG comprises:
preparing a d-2-HG solution in gradient concentrations using healthy adult serum, urine, and cell culture medium as different types of biological samples, respectively, determining dose-response curves and quantitative results for d-2-HG in the different types of biological samples by using the d-2-HG biosensor B_{d2HG}-1;
wherein the method of determining dose-response curves for d-2-HG in different types of biological samples comprises: adding 1 nM biotinylated dhdO-1 fragment, 0.3 nM DhdR protein, and equal volumes of a solution containing different concentrations of D-2-HG into a white 384-well plate, mixing evenly and incubating for 30 minutes; adding 20 μg/mL acceptor beads and incubating for 30 minutes; adding 20 μg/mL donor beads and incubating for 60 minutes, wherein the incubation is performed at room temperature and in dark;
measuring the luminescence signal for each sample by EnSight Multimode Plate Reader, wherein the excitation wavelength is 680 nm, the detection wavelength is 520-620 nm and subtracting the background signal without D-2-HG biosensor at each emission wavelength, and obtaining the dose-response curves for D-2-HG in different types of biological samples;
wherein the method for quantification of D-2-HG concentrations in different types of biological samples comprises: determining the luminescence signals of different types of samples at 50 μM, 150 μM, 500 μM, 1500 μM, and 3500 μM using the method of determining the dose-response curves, subtracting the background signal without d-2-HG biosensor at each emission wavelength; using the dose-response curves of d-2-HG in different types of biological samples to correspond to the luminescence signal values of the samples after subtracting the background signals to the concentrations of d-2-HG and multiplying them by the corresponding dilutions to obtain the quantitative results of d-2-HG in different types of biological samples.

9. The application of the biosensor for detecting d-2-HG according to claim 4 in detecting biological samples containing d-2-HG.
